(19) United States
(12) Patent Application Publication
(10) Pub. No.: US 2020/0060597 A1
(43) Pub. Date: Feb. 27, 2020

(54) STATE ESTIMATION DEVICE

(71) Applicant: MITSUBISHI ELECTRIC CORPORATION, Tokyo (JP)

(72) Inventors: Isamu OGAWA, Tokyo (JP); Takahiro OTSUKA, Tokyo (JP)

(73) Assignee: MITSUBISHI ELECTRIC CORPORATION, Tokyo (JP)

(21) Appl. No.: 16/344,091

(22) PCT Filed: Dec. 14, 2016
(86) PCT No.: PCT/JP2016/087204

§ 371 Date: Apr. 23, 2019

(51) Int. Cl. A61B 5/16; A61B 5/11; A61B 5/00; G01K 13/00; G10L 25/63; G06K 9/00

(52) U.S. Cl. CPC A61B 5/162; A61B 5/11; A61B 5/7278; A61B 2562/0204; G10L 25/63; G06K 9/00335; A61B 2560/0242; G01K 13/00

(57) ABSTRACT

A state estimation device includes an action detecting unit that checks behavioral information against action patterns stored in advance, and detects a matching action pattern; a reaction detecting unit that checks the behavioral and biological information about a user against reaction patterns stored, and detects a matching reaction pattern; a discomfort determining unit that determines that the user is in an uncomfortable state, when a matching action pattern is detected, or a matching reaction pattern is detected and the detected reaction pattern matches a discomfort reaction pattern; a discomfort zone estimating unit that acquires an estimation condition, and estimates a discomfort zone; and a learning unit that refers to the history information, and acquires and stores the discomfort reaction pattern based on the estimated discomfort zone and the occurrence frequencies of the reaction patterns in a zone other than the discomfort zone.

### TECHNICAL FIELD

The present invention relates to a technique for estimating an emotional state of a user.

### BACKGROUND ART

There have been techniques for estimating an emotional state of a user from biological information acquired from a wearable sensor or the like. The estimated emotion of the user is referred to as information for providing a recommended service depending on a state of the user, for example.

For example, Patent Literature 1 discloses an emotional information estimating device that performs machine learning to generate an estimator that learns the relationship between biological information and emotional information on the basis of a history accumulation database that stores a user's biological information acquired beforehand and the user's emotional information and physical states corresponding to the biological information, and estimates emotional information from the biological information for each physical state. The emotional information estimating device estimates emotional information of the user from the user's biological information detected with the estimator corresponding to the physical state of the user.

### CITATION LIST

### Patent Literature

Patent Literature 1: JP 2013-73985 A

### SUMMARY OF INVENTION

### Technical Problem

In the above described emotional information estimating device of Patent Literature 1, to build the history accumulation database, the user needs to input his/her emotional information corresponding to biological information. Therefore, a great burden is put on the user in performing input operations, and user-friendliness becomes lower.

Furthermore, to obtain a high-precision estimator through machine learning, any estimator cannot be used until a sufficiently large amount of information is accumulated in the history accumulation database.

The present invention has been made to solve the above problems, and aims to estimate an emotional state of a user, without the user inputting his/her emotional state, even in a case where information indicating emotional states of the user and information indicating physical states are not accumulated.

### Solution to Problem

A state estimation device according to this invention includes: an action detecting unit that checks at least one piece of behavioral information including motion information about a user, sound information about the user, and operation information about the user against action patterns stored in advance, and detects a matching action pattern; a reaction detecting unit that checks the behavioral information and biological information about the user against reaction patterns stored in advance, and detects a matching reaction pattern; a discomfort determining unit that determines that the user is in an uncomfortable state, when the action detecting unit has detected a matching action pattern, or when the reaction detecting unit has detected a matching reaction pattern and the detected reaction pattern matches a discomfort reaction pattern indicating an uncomfortable state of the user, the discomfort reaction pattern being stored in advance; a discomfort zone estimating unit that acquires an estimation condition for estimating a discomfort zone on the basis of the action pattern detected by the action detecting unit, and estimates a discomfort zone that is a zone matching the acquired estimation condition in history information stored in advance; and a learning unit that acquires and stores the discomfort reaction pattern on the basis of the discomfort zone estimated by the discomfort zone estimating unit and the occurrence frequency of a reaction pattern in a zone other than the discomfort zone, by referring to the history information.

### Advantageous Effects of Invention

According to this invention, it is possible to estimate an emotional state of a user, without the user inputting his/her emotional state, even in a case where information indicating emotional states of the user and information indicating physical states are not accumulated.

### BRIEF DESCRIPTION OF DRAWINGS

FIG. 1 is a block diagram showing the configuration of a state estimation device according to a first embodiment.

FIG. 2 is a table showing an example of storage in an action information database of the state estimation device according to the first embodiment.

FIG. 3 is a table showing an example of the storage in a reaction information database of the state estimation device according to the first embodiment.

FIG. 4 is a table showing an example of the storage in a discomfort reaction pattern database of the state estimation device according to the first embodiment.

FIG. 5 is a table showing an example of the storage in a learning database of the state estimation device according to the first embodiment.

FIGS. 6A and 6B are diagrams each showing an example hardware configuration of the state estimation device according to the first embodiment.

FIG. 7 is a flowchart showing an operation of the state estimation device according to the first embodiment.

FIG. 8 is a flowchart showing an operation of an environmental information acquiring unit of the state estimation device according to the first embodiment.

FIG. 9 is a flowchart showing an operation of a behavioral information acquiring unit of the state estimation device according to the first embodiment.

FIG. 10 is a flowchart showing an operation of a biological information acquiring unit of the state estimation device according to the first embodiment.

FIG. 11 is a flowchart showing an operation of an action detecting unit of the state estimation device according to the first embodiment.

FIG. 12 is a flowchart showing an operation of a reaction detecting unit of the state estimation device according to the first embodiment.

FIG. 13 is a flowchart showing operations of a discomfort determining unit, a discomfort reaction pattern learning unit, and a discomfort zone estimating unit of the state estimation device according to the first embodiment.

FIG. 14 is a flowchart showing an operation of the discomfort reaction pattern learning unit of the state estimation device according to the first embodiment.

FIG. 15 is a flowchart showing an operation of the discomfort zone estimating unit of the state estimation device according to the first embodiment.

FIG. 16 is a flowchart showing an operation of the discomfort reaction pattern learning unit of the state estimation device according to the first embodiment.

FIG. 17 is a flowchart showing an operation of the discomfort reaction pattern learning unit of the state estimation device according to the first embodiment.

FIG. 18 is a diagram showing an example of learning of discomfort reaction patterns in the state estimation device according to the first embodiment.

FIG. 19 is a flowchart showing an operation of the discomfort determining unit of the state estimation device according to the first embodiment.

FIG. 20 is a diagram showing an example of uncomfortable state estimation by the state estimation device according to the first embodiment.

FIG. 21 is a block diagram showing the configuration of a state estimation device according to a second embodiment.

FIG. 22 is a flowchart showing an operation of an estimator generating unit of the state estimation device according to the second embodiment.

FIG. 23 is a flowchart showing an operation of a discomfort determining unit of the state estimation device according to the second embodiment.

FIG. 24 is a block diagram showing the configuration of a state estimation device according to a third embodiment.

FIG. 25 is a table showing an example of storage in a discomfort reaction pattern database of the state estimation device according to the third embodiment.

FIG. 26 is a flowchart showing an operation of a discomfort determining unit of the state estimation device according to the third embodiment.

FIG. 27 is a flowchart showing an operation of the discomfort determining unit of the state estimation device according to the third embodiment.

### DESCRIPTION OF EMBODIMENTS

To explain the present invention in greater detail, modes for carrying out the invention are described below with reference to the accompanying drawings.

### First Embodiment

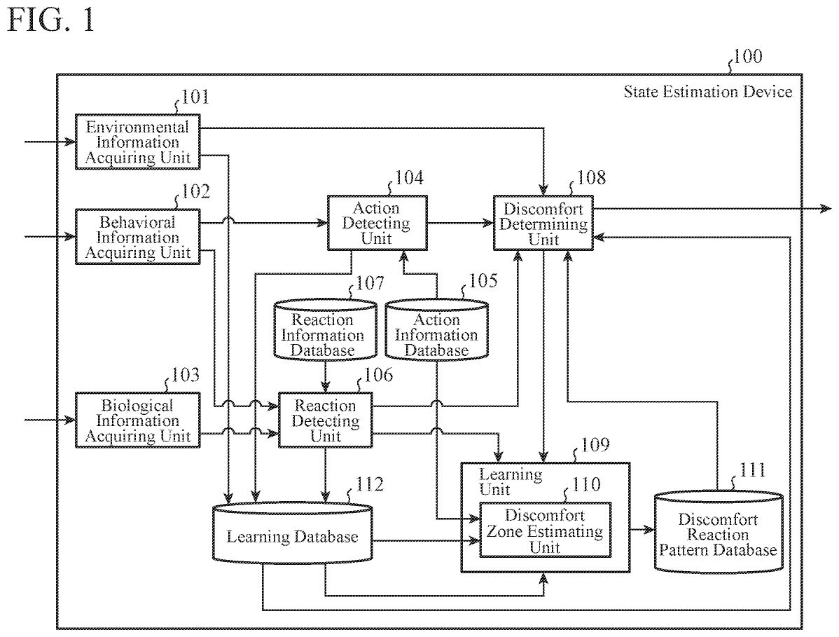

FIG. 1 is a block diagram showing the configuration of a state estimation device 100 according to a first embodiment.

The state estimation device 100 includes an environmental information acquiring unit 101, a behavioral information acquiring unit 102, a biological information acquiring unit 103, an action detecting unit 104, an action information database 105, a reaction detecting unit 106, a reaction information database 107, a discomfort determining unit 108, a learning unit 109, a discomfort zone estimating unit 110, a discomfort reaction pattern database 111, and a learning database 112.

The environmental information acquiring unit 101 acquires information about the temperature around a user and noise information indicating the magnitude of noise as environmental information. The environmental information acquiring unit 101 acquires information detected by a temperature sensor, for example, as the temperature information. The environmental information acquiring unit 101 acquires information indicating the magnitude of sound collected by a microphone, for example, as the noise information. The environmental information acquiring unit 101 outputs the acquired environmental information to the discomfort determining unit 108 and the learning database 112.

The behavioral information acquiring unit 102 acquires behavioral information that is motion information indicating movement of a user's face and body, sound information indicating the user's utterance and the sound emitted by the user, and operation information indicating operation of the user's device.

The behavioral information acquiring unit 102 acquires, as the motion information, information indicating the expression of a user, movement of part of the face of the user, motion of the user's body part such as the head, a hand, an arm, a leg, or the chest. This information is obtained through analysis of an image captured by a camera, for example.

The behavioral information acquiring unit 102 acquires, as the sound information, a voice recognition result indicating the content of a user's utterance obtained through analysis of sound signals collected by a microphone, for example, and a sound recognition result indicating the sound uttered by the user (such as the sound of clicking of the user's tongue).

The behavioral information acquiring unit 102 acquires, as the operation information, information about a user operating a device detected by a touch panel or a physical switch (such as information indicating that a button for raising the sound volume has been pressed).

The behavioral information acquiring unit 102 outputs the acquired behavioral information to the action detecting unit 104 and the reaction detecting unit 106.

The biological information acquiring unit 103 acquires information indicating fluctuations in the heart rate of a user as biological information. The biological information acquiring unit 103 acquires, as the biological information, information indicating fluctuations in the heart rate of a user measured by a heart rate meter or the like the user is wearing, for example. The biological information acquiring unit 103 outputs the acquired biological information to the reaction detecting unit 106.

The action detecting unit 104 checks the behavioral information input from the behavioral information acquiring unit 102 against the action patterns in the action information stored in the action information database 105. In a case where an action pattern matching the behavioral information is stored in the action information database 105, the action detecting unit 104 acquires the identification information about the action pattern. The action detecting unit 104 outputs the acquired identification information about the action pattern to the discomfort determining unit 108 and the learning database 112.

The action information database 105 is a database that defines and stores action patterns of users for respective discomfort factors.

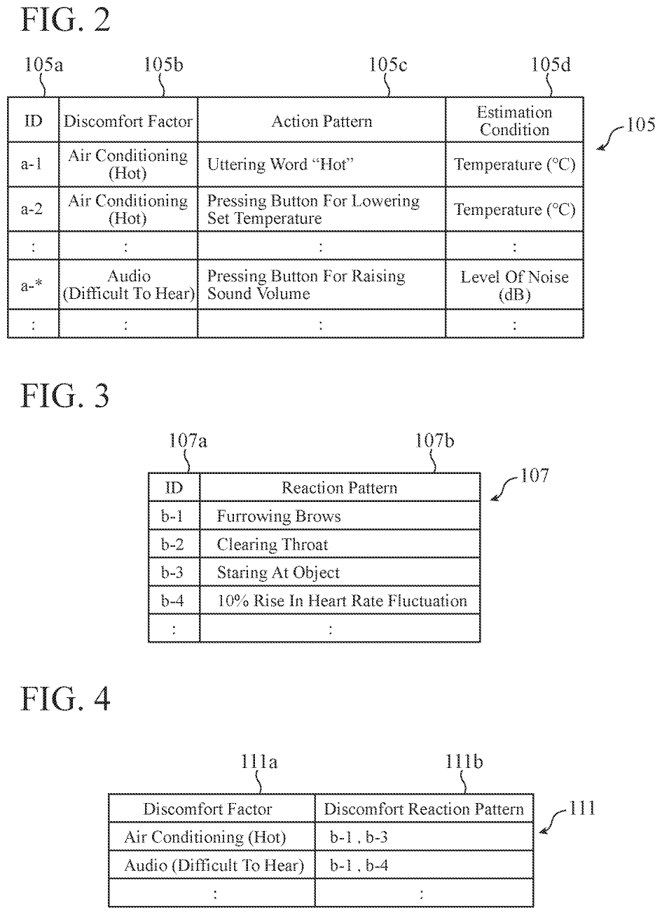

FIG. 2 is a table showing an example of the storage in the action information database 105 of the state estimation device 100 according to the first embodiment.

The action information database 105 shown in FIG. 2 contains the following items: IDs 105a, discomfort factors 105b, action patterns 105c, and estimation conditions 105d.

In the action information database 105, an action pattern 105c is defined for each one discomfort factor 105b. An estimation condition 105d that is a condition for estimating a discomfort zone is set for each one action pattern 105c. An ID 105a as identification information is also attached to each one action pattern 105c.

Action patterns of users associated directly with the discomfort factors 105b are set as the action patterns 105c. In the example shown in FIG. 2, “uttering the word “hot”” and “pressing the button for lowering the set temperature” are set as the action patterns of users associated directly with a discomfort factor 105b that is “air conditioning (hot)”.

The reaction detecting unit 106 checks the behavioral information input from the behavioral information acquiring unit 102 and the biological information input from the biological information acquiring unit 103 against the reaction information stored in the reaction information database 107. In a case where a reaction pattern matching the behavioral information or the biological information is stored in the reaction information database 107, the reaction detecting unit 106 acquires the identification information associated with the reaction pattern. The reaction detecting unit 106 outputs the acquired identification information about the reaction pattern to the discomfort determining unit 108, the learning unit 109, and the learning database 112.

The reaction information database 107 is a database that stores reaction patterns of users.

FIG. 3 is a table showing an example of the storage in the reaction information database 107 of the state estimation device 100 according to the first embodiment.

The reaction information database 107 shown in FIG. 3 contains the following items: IDs 107a and reaction patterns 107b. An ID 107a as identification information is attached to each one reaction pattern 107b.

Reaction patterns of users not associated directly with discomfort factors (the discomfort factors 105b shown in FIG. 2, for example) are set as the reaction patterns 107b. In the example shown in FIG. 3, “furrowing brows” and “clearing throat” are set as reaction patterns observed when a user is in an uncomfortable state.

When the identification information about the detected action pattern is input from the action detecting unit 104, the discomfort determining unit 108 outputs, to the outside, a signal indicating that the uncomfortable state of the user has been detected. The discomfort determining unit 108 also outputs the input identification information about the action pattern to the learning unit 109, and instructs the learning unit 109 to learn reaction patterns.

Further, when the identification information about the detected reaction pattern is input from the reaction detecting unit 106, the discomfort determining unit 108 checks the input identification information against the discomfort reaction patterns that are stored in the discomfort reaction pattern database 111 and indicate uncomfortable states of users. In a case where a reaction pattern matching the input identification information is stored in the discomfort reaction pattern database 111, the discomfort determining unit 108 estimates that the user is in an uncomfortable state. The discomfort determining unit 108 outputs, to the outside, a signal indicating that the user's uncomfortable state has been detected.

The discomfort reaction pattern database 111 will be described later in detail.

As shown in FIG. 1, the learning unit 109 includes the discomfort zone estimating unit 110. When a reaction pattern learning instruction is issued from the discomfort determining unit 108, the discomfort zone estimating unit 110 acquires an estimation condition for estimating a discomfort zone from the action information database 105, using the action pattern identification information that has been input at the same time as the instruction. The discomfort zone estimating unit 110 acquires the estimation condition 105d corresponding to the ID 105a that is the identification information about the action pattern shown in FIG. 2, for example. By referring to the learning database 112, the discomfort zone estimating unit 110 estimates a discomfort zone from the information matching the acquired estimation condition.

By referring to the learning database 112, the learning unit 109 extracts the identification information about one or more reaction patterns in the discomfort zone estimated by the discomfort zone estimating unit 110. On the basis of the extracted identification information, the learning unit 109 further refers to the learning database 112, to extract the reaction patterns generated in the past at frequencies equal to or higher than a threshold as discomfort reaction pattern candidates.

By referring to the learning database 112, the learning unit 109 further extracts the reaction patterns generated at frequencies equal to or higher than the threshold in the zones other than the discomfort zone estimated by the discomfort zone estimating unit 110 as patterns that are not discomfort reaction patterns (these patterns will be hereinafter referred to as non-discomfort reaction patterns). The learning unit 109 excludes the extracted non-discomfort reaction patterns from the discomfort reaction pattern candidates.

The learning unit 109 stores a combination of identification information about the eventually remaining discomfort reaction pattern candidates as a discomfort reaction pattern into the discomfort reaction pattern database 111 for each discomfort factor.

The discomfort reaction pattern database 111 is a database that stores discomfort reaction patterns that are the results of learning by the learning unit 109.

FIG. 4 is a table showing an example of the storage in the discomfort reaction pattern database 111 of the state estimation device 100 according to the first embodiment.

The discomfort reaction pattern database 111 shown in FIG. 4 contains the following items: discomfort factors 111a and discomfort reaction patterns 111b. The same items as the items of the discomfort factors 105b in the action information database 105 are written as the discomfort factors 111a.

The IDs 107a corresponding to the reaction patterns 107b in the reaction information database 107 are written as the discomfort reaction patterns 111b.

In a case where the discomfort factor is “air conditioning (hot)” in FIG. 4, the user shows the reactions “furrowing brows” of ID “b-1” and “staring at the object” of ID “b-3”.

The learning database 112 is a database that stores results of learning of action patterns and reaction patterns when the environmental information acquiring unit 101 acquires environmental information.

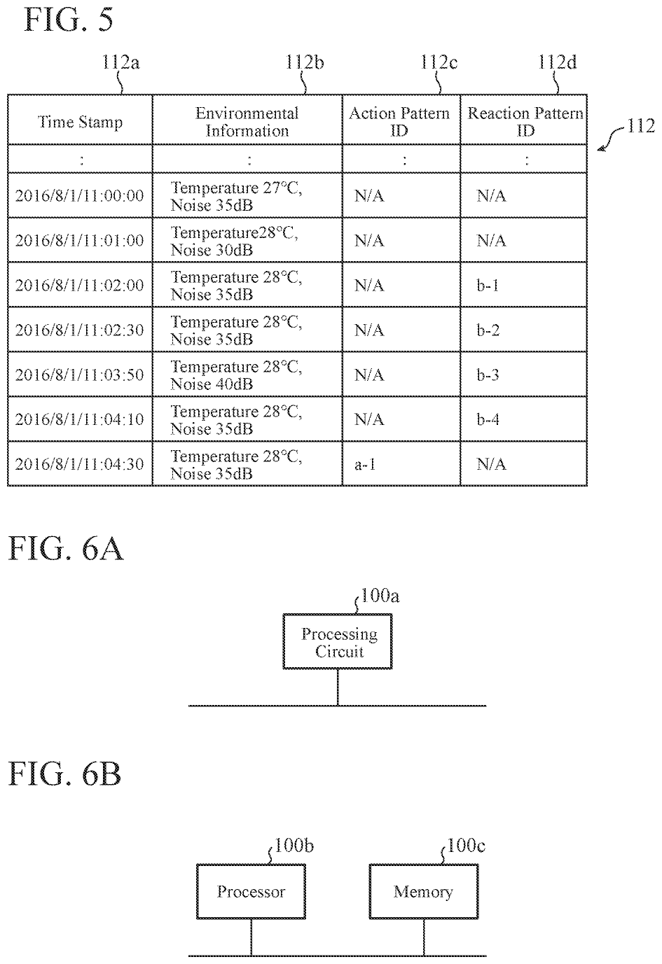

FIG. 5 is a table showing an example of the storage in the learning database 112 of the state estimation device 100 according to the first embodiment.

The learning database 112 shown in FIG. 5 contains the following items: time stamps 112a, environmental information 112b, action pattern IDs 112c, and reaction pattern IDs 112d.

The time stamps 112a are information indicating the times at which the environmental information 112b has been acquired.

The environmental information 112b is temperature information, noise information, and the like at the times indicated by the time stamps 112a. The action pattern IDs 112c are the identification information acquired by the action detecting unit 104 at the times indicated by the time stamps 112a. The reaction pattern IDs 112d are the identification information acquired by the reaction detecting unit 106 at the times indicated by the time stamps 112a.

As shown in FIG. 5, when the time stamp 112a is “2016/8/1/11:02:00”, the environmental information 112b is “temperature 28° C., noise 35 dB”, the action detecting unit 104 detected no action patterns indicating the user's discomfort, and the reaction detecting unit 106 detected the reaction pattern of “furrowing brows” of ID “b-1”.

Next, an example hardware configuration of the state estimation device 100 is described.

FIGS. 6A and 6B are diagrams each showing an example hardware configuration of the state estimation device 100.

The environmental information acquiring unit 101, the behavioral information acquiring unit 102, the biological information acquiring unit 103, the action detecting unit 104, the reaction detecting unit 106, the discomfort determining unit 108, the learning unit 109, and the discomfort zone estimating unit 110 in the state estimation device 100 may be a processing circuit 100a that is dedicated hardware as shown in 6A, or may be a processor 100b that executes a program stored in a memory 100c as shown in FIG. 6B.

As shown in FIG. 6A, in a case where the environmental information acquiring unit 101, the behavioral information acquiring unit 102, the biological information acquiring unit 103, the action detecting unit 104, the reaction detecting unit 106, the discomfort determining unit 108, the learning unit 109, and the discomfort zone estimating unit 110 are dedicated hardware, the processing circuit 100a may be a single circuit, a composite circuit, a programmed processor, a parallel-programmed processor, an application specific integrated circuit (ASIC), a field-programmable gate array (FPGA), or a combination of the above, for example. Each of the functions of the respective components of the environmental information acquiring unit 101, the behavioral information acquiring unit 102, the biological information acquiring unit 103, the action detecting unit 104, the reaction detecting unit 106, the discomfort determining unit 108, the learning unit 109, and the discomfort zone estimating unit 110 may be formed with a processing circuit, or the functions of the respective components may be collectively formed with one processing circuit.

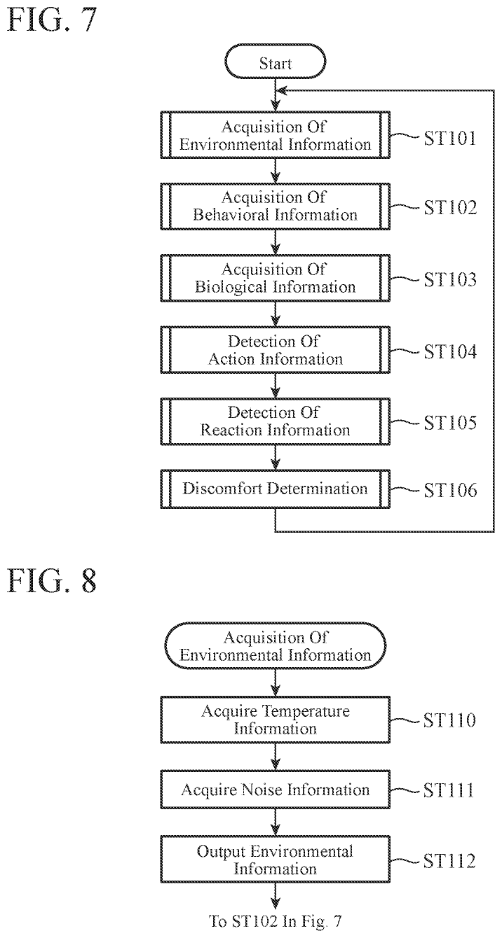

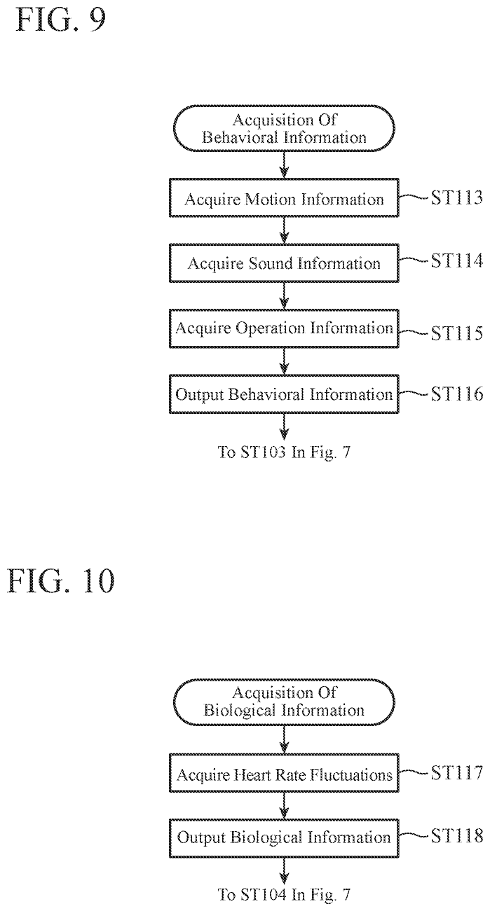

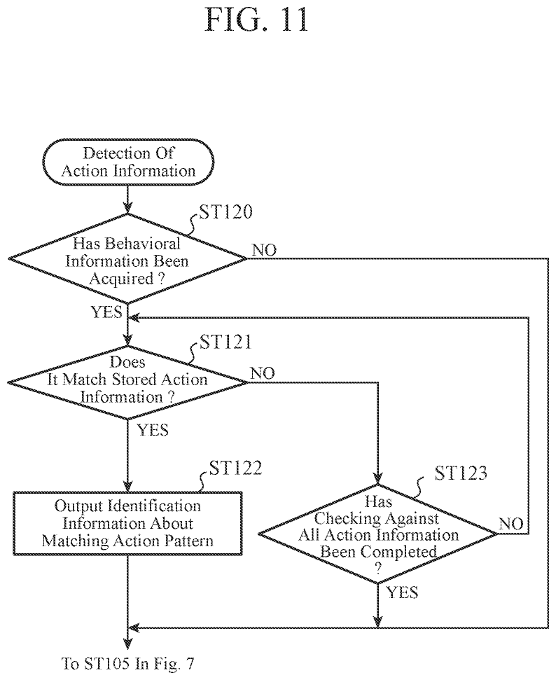

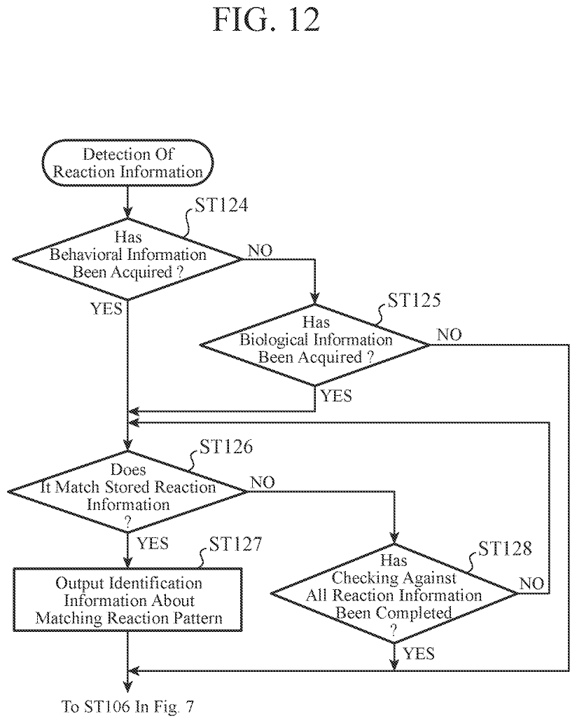

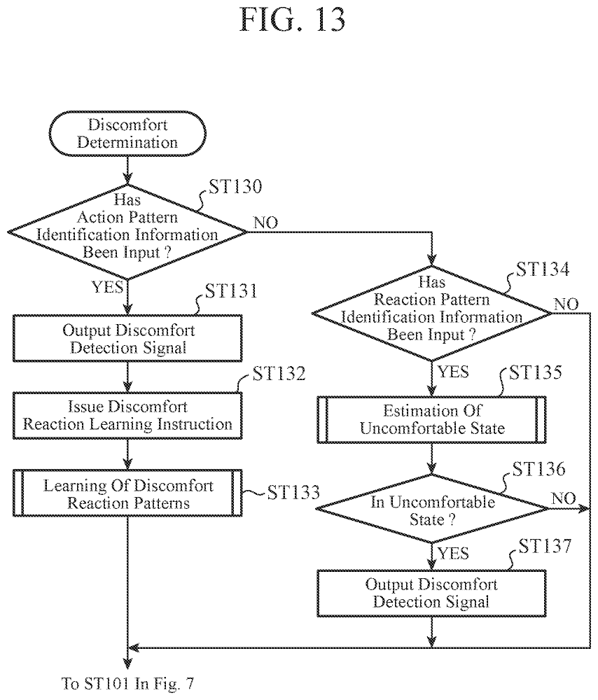

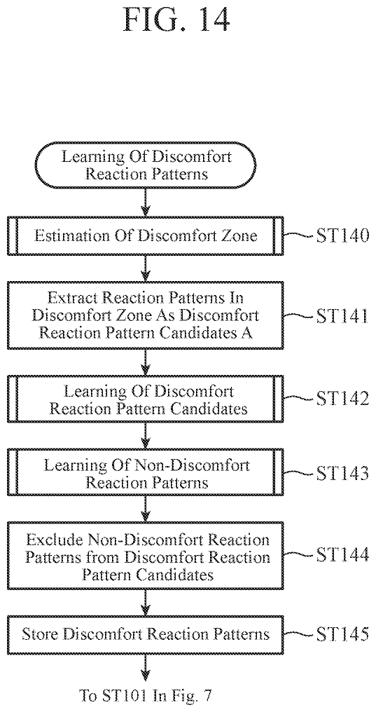

As shown in FIG. 6B, in a case where the environmental information acquiring unit 101, the behavioral information acquiring unit 102, the biological information acquiring unit 103, the action detecting unit 104, the reaction detecting unit 106, the discomfort determining unit 108, the learning unit 109, and the discomfort zone estimating unit 110 are the processor 100b, the functions of the respective components are formed with software, firmware, or a combination of software and firmware. Software or firmware is written as programs, and is stored in the memory 100c. By reading and executing the programs stored in the memory 100c, the processor 100b achieves the respective functions of the environmental information acquiring unit 101, the behavioral information acquiring unit 102, the biological information acquiring unit 103, the action detecting unit 104, the reaction detecting unit 106, the discomfort determining unit 108, the learning unit 109, and the discomfort zone estimating unit 110. That is, the environmental information acquiring unit 101, the behavioral information acquiring unit 102, the biological information acquiring unit 103, the action detecting unit 104, the reaction detecting unit 106, the discomfort determining unit 108, the learning unit 109, and the discomfort zone estimating unit 110 have the memory 100c for storing the programs by which the respective steps shown in FIGS. 7 through 17 and FIG. 19, which will be described later, are eventually carried out when executed by the processor 100b. It can also be said that these programs are for causing a computer to implement procedures or a method involving the environmental information acquiring unit 101, the behavioral information acquiring unit 102, the biological information acquiring unit 103, the action detecting unit 104, the reaction detecting unit 106, the discomfort determining unit 108, the learning unit 109, and the discomfort zone estimating unit 110.

Here, the processor 100b is a central processing unit (CPU), a processing device, an arithmetic device, a processor, a microprocessor, a microcomputer, a digital signal processor (DSP), or the like, for example.

The memory 100c may be a nonvolatile or volatile semiconductor memory such as a random access memory (RAM), a read only memory (ROM), a flash memory, an erasable programmable ROM (EPROM), or an electrically EPROM (EEPROM), may be a magnetic disk such as a hard disk or a flexible disk, or may be an optical disc such as a mini disc, a compact disc (CD), or a digital versatile disc (DVD), for example.

Note that some of the functions of the environmental information acquiring unit 101, the behavioral information acquiring unit 102, the biological information acquiring unit 103, the action detecting unit 104, the reaction detecting unit 106, the discomfort determining unit 108, the learning unit 109, and the discomfort zone estimating unit 110 may be formed with dedicated hardware, and the other functions may be formed with software or firmware. In this manner, the processing circuit 100a in the state estimation device 100 can achieve the above described functions with hardware, software, firmware, or a combination thereof.

Next, operation of the state estimation device 100 is described.

FIG. 7 is a flowchart showing an operation of the state estimation device 100 according to the first embodiment.

The environmental information acquiring unit 101 acquires environmental information (step ST101).

FIG. 8 is a flowchart showing an operation of the environmental information acquiring unit 101 of the state estimation device 100 according to the first embodiment, and is a flowchart showing the process in step ST101 in detail.

The environmental information acquiring unit 101 acquires information detected by a temperature sensor, for example, as temperature information (step ST110). The environmental information acquiring unit 101 acquires information indicating the magnitude of sound collected by a microphone, for example, as noise information (step ST111). The environmental information acquiring unit 101 outputs the temperature information acquired in step ST110 and the noise information acquired in step ST111 as environmental information to the discomfort determining unit 108 and the learning database 112 (step ST112).

By the processes in steps ST110 through ST112 described above, information is stored as items of a time stamp 112a and environmental information 112b in the learning database 112 shown in FIG. 5, for example. After that, the flowchart proceeds to the process in step ST102 in FIG. 7.

In the flowchart in FIG. 7, the behavioral information acquiring unit 102 then acquires behavioral information about the user (step ST102).

FIG. 9 is a flowchart showing an operation of the behavioral information acquiring unit 102 of the state estimation device 100 according to the first embodiment, and is a flowchart showing the process in step ST102 in detail.

The behavioral information acquiring unit 102 acquires motion information obtained by analyzing a captured image, for example (step ST113). The behavioral information acquiring unit 102 acquires sound information obtained by analyzing a sound signal, for example (step ST114). The behavioral information acquiring unit 102 acquires information about operation of a device, for example, as operation information (step ST115). The behavioral information acquiring unit 102 outputs the motion information acquired in step ST113, the sound information acquired in step ST114, and the operation information acquired in step ST115 as behavioral information to the action detecting unit 104 and the reaction detecting unit 106 (step ST116). After that, the flowchart proceeds to the process in step ST103 in FIG. 7.

In the flowchart in FIG. 7, the biological information acquiring unit 103 then acquires biological information about the user (step ST103).

FIG. 10 is a flowchart showing an operation of the biological information acquiring unit 103 of the state estimation device 100 according to the first embodiment, and is a flowchart showing the process in step ST103 in detail.

The biological information acquiring unit 103 acquires information indicating fluctuations in the heart rate of the user, for example, as biological information (step ST117). The biological information acquiring unit 103 outputs the biological information acquired in step ST117 to the reaction detecting unit 106 (step ST118). After that, the flowchart proceeds to the process in step ST104 in FIG. 7.

In the flowchart in FIG. 7, the action detecting unit 104 then detects action information about the user from the behavioral information input from the behavioral information acquiring unit 102 in step ST102 (step ST104).

FIG. 11 is a flowchart showing an operation of the action detecting unit 104 of the state estimation device 100 according to the first embodiment, and is a flowchart showing the process in step ST104 in detail.

The action detecting unit 104 determines whether behavioral information has been input from the behavioral information acquiring unit 102 (step ST120). If any behavioral information has not been input (step ST120; NO), the process comes to an end, and the operation proceeds to the process in step ST105 in FIG. 7. If behavioral information has been input (step ST120; YES), on the other hand, the action detecting unit 104 determines whether the input behavioral information matches an action pattern in the action information stored in the action information database 105 (step ST121).

If the input behavioral information matches an action pattern in the action information stored in the action information database 105 (step ST121; YES), the action detecting unit 104 acquires the identification information attached to the matching action pattern, and outputs the identification information to the discomfort determining unit 108 and the learning database 112 (step ST122). If the input behavioral information does not match any action pattern in the action information stored in the action information database 105 (step ST121; NO), on the other hand, the action detecting unit 104 determines whether checking against all the action information has been completed (step ST123). If checking against all the action information has not been completed yet (step ST123; NO), the operation returns to the process in step ST121, and the above described processes are repeated. If the process in step ST122 has been performed, or if checking against all the action information has been completed (step ST123; YES), on the other hand, the flowchart proceeds to the process in step ST105 in FIG. 7.

In the flowchart in FIG. 7, the reaction detecting unit 106 then detects reaction information about the user (step ST105). Specifically, the reaction detecting unit 106 detects reaction information about the user, using the behavioral information input from the behavioral information acquiring unit 102 in step ST102 and the biological information input from the biological information acquiring unit 103 in step ST103.

FIG. 12 is a flowchart showing an operation of the reaction detecting unit 106 of the state estimation device 100 according to the first embodiment, and is a flowchart showing the process in step ST105 in detail.

The reaction detecting unit 106 determines whether behavioral information has been input from the behavioral information acquiring unit 102 (step ST124). If any behavioral information has not been input (step ST124; NO), the reaction detecting unit 106 determines whether biological information has been input from the biological information acquiring unit 103 (step ST125). If any biological information has not been input (step ST125; NO), the process comes to an end, and the operation proceeds to the process in step ST106 in the flowchart shown in FIG. 7.

If behavioral information has been input (step ST124; YES), or if biological information has been input (step ST125; YES), on the other hand, the reaction detecting unit 106 determines whether the input behavioral information or biological information matches a reaction pattern in the reaction information stored in the reaction information database 107 (step ST126). If the input behavioral information or biological information matches a reaction pattern in the reaction information stored in the reaction information database 107 (step ST126; YES), the reaction detecting unit 106 acquires the identification information attached to the matching reaction pattern, and outputs the identification information to the discomfort determining unit 108, the learning unit 109, and the learning database 112 (step ST127).

If the input behavioral information or biological information does not match any reaction pattern in the reaction information stored in the reaction information database 107 (step ST126; NO), the reaction detecting unit 106 determines whether checking against all the reaction information has been completed (step ST128). If checking against all the reaction information has not been completed yet (step ST128; NO), the operation returns to the process in step ST126, and the above described processes are repeated. If the process in step ST127 has been performed, or if checking against all the reaction information has been completed (step ST128; YES), on the other hand, the flowchart proceeds to the process in step ST106 in FIG. 7.

When the action information detecting process by the action detecting unit 104 and the reaction information detecting process by the reaction detecting unit 106 are completed in the flowchart in FIG. 7, the discomfort determining unit 108 then determines whether the user is in an uncomfortable state (step ST106).

FIG. 13 is a flowchart showing operations of the discomfort determining unit 108, the learning unit 109, and the discomfort zone estimating unit 110 of the state estimation device 100 according to the first embodiment, and is a flowchart showing the process in step ST106 in detail.

The discomfort determining unit 108 determines whether identification information about an action pattern has been input from the action detecting unit 104 (step ST130). If identification information about an action pattern has been input (step ST130; YES), the discomfort determining unit 108 outputs, to the outside, a signal indicating that an uncomfortable state of the user has been detected (step ST131). The discomfort determining unit 108 also outputs the input identification information about the action pattern to the learning unit 109, and instructs the learning unit 109 to learn discomfort reaction patterns (step ST132). The learning unit 109 learns a discomfort reaction pattern on the basis of the action pattern identification information and the learning instruction input in step ST132 (step ST133). The process of learning discomfort reaction patterns in step ST133 will be described later in detail.

If any identification information about any action pattern has not been input (step ST130; NO), on the other hand, the discomfort determining unit 108 determines whether identification information about a reaction pattern has been input from the reaction detecting unit 106 (step ST134). If identification information about a reaction pattern has been input (step ST134; YES), the discomfort determining unit 108 checks the reaction pattern indicated by the identification information against the discomfort reaction patterns stored in the discomfort reaction pattern database 111, and estimates an uncomfortable state of the user (step ST135). The process of estimating an uncomfortable state in step ST135 will be described later in detail.

The discomfort determining unit 108 refers to the result of the estimation in step ST135, and determines whether the user is in an uncomfortable state (step ST136). If the user is determined to be in an uncomfortable state (step ST136; YES), the discomfort determining unit 108 outputs a signal indicating that the user's uncomfortable state has been detected, to the outside (step ST137). In the process in step ST137, the discomfort determining unit 108 may add information indicating a discomfort factor to the signal to be output to the outside.

If the process in step ST133 has been performed, if the process in step ST137 has been performed, if any identification information about any reaction pattern has not been input (step ST134; NO), or if the user is determined not to be in an uncomfortable state (step ST136; NO), the flowchart returns to the process in step ST101 in FIG. 7.

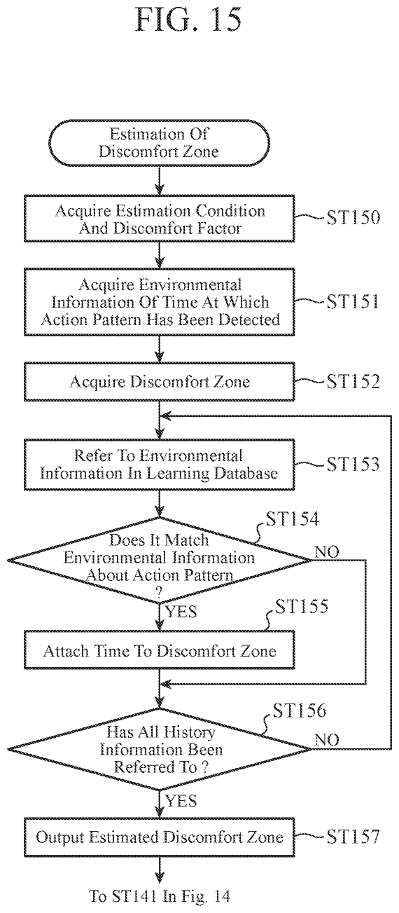

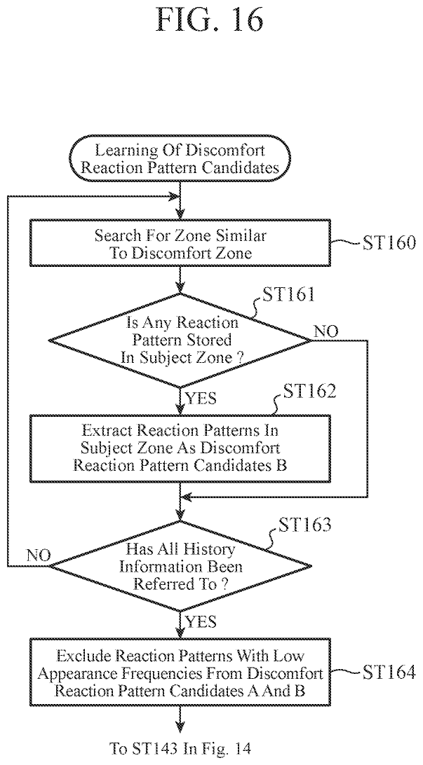

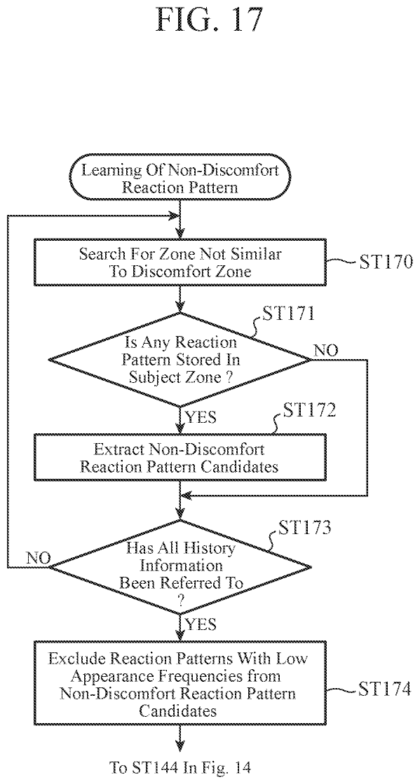

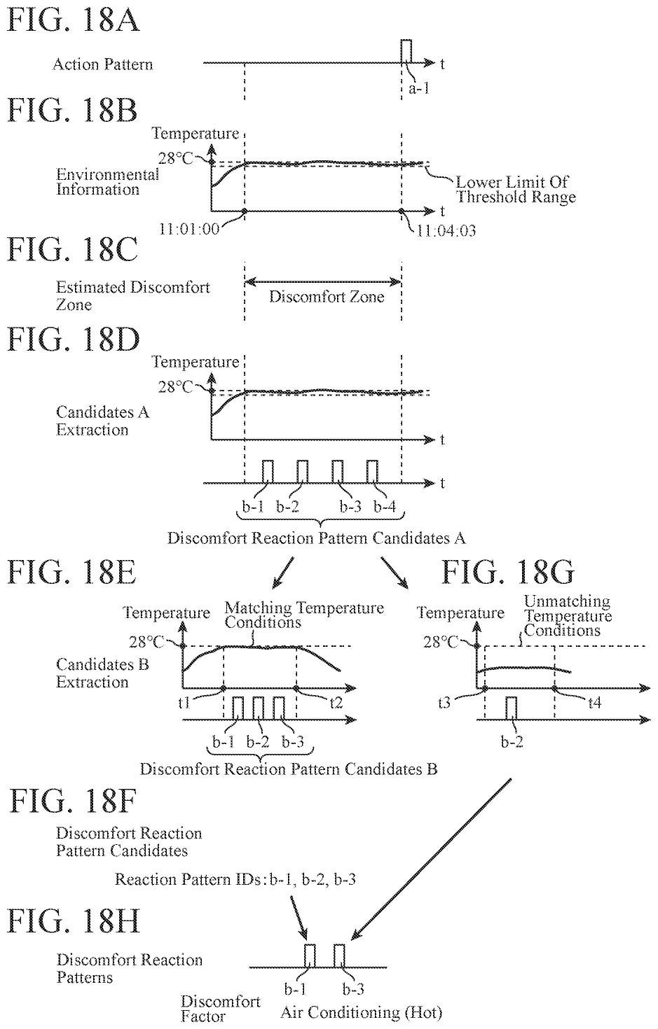

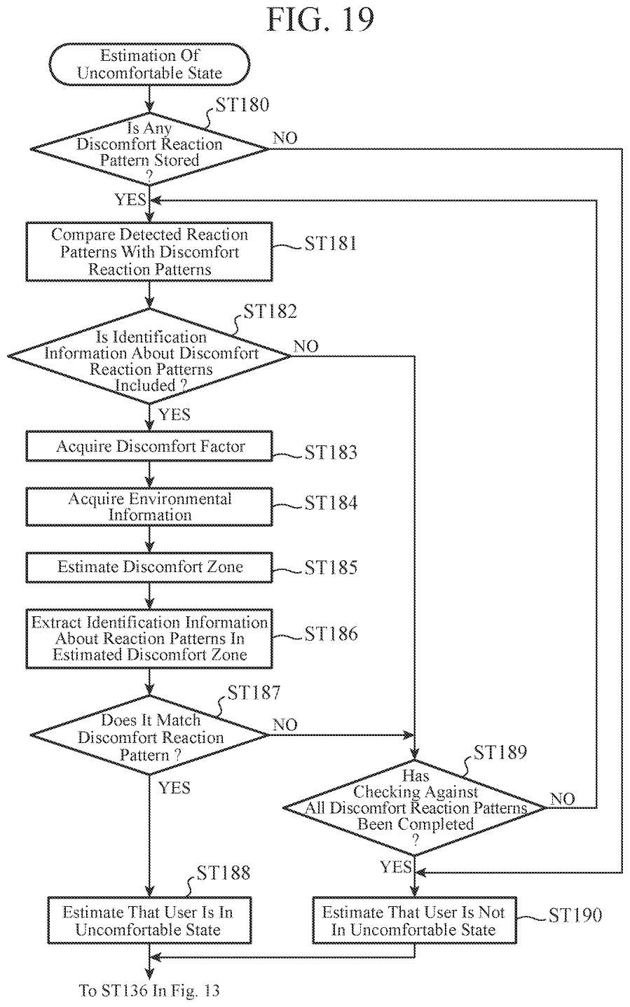

Next, the above mentioned process in step ST133 in the flowchart in FIG. 13 is described in detail. The following description will be made with reference to the storage examples shown in FIGS. 2 through 5, flowcharts shown in FIGS. 14 through 17, and an example of discomfort reaction pattern learning shown in FIG. 18.

FIG. 14 is a flowchart showing an operation of the learning unit 109 of the state estimation device 100 according to the first embodiment.

FIG. 18 is a diagram showing an example of learning of discomfort reaction patterns in the state estimation device 100 according to the first embodiment.

In the flowchart in FIG. 14, the discomfort zone estimating unit 110 of the learning unit 109 estimates a discomfort zone from the action pattern identification information input from the discomfort determining unit 108 (step ST140).

FIG. 15 is a flowchart showing an operation of the discomfort zone estimating unit 110 of the state estimation device 100 according to the first embodiment, and is a flowchart showing the process in step ST140 in detail.

Using the action pattern identification information input from the discomfort determining unit 108, the discomfort zone estimating unit 110 searches the action information database 105, and acquires the estimation condition and the discomfort factor associated with the action pattern (step ST150).

For example, as shown in FIG. 18A, in a case where the action pattern indicated by the identification information (ID; a-1) is input, the discomfort zone estimating unit 110 searches the action information database 105 shown in FIG. 2, and acquires the estimation condition “temperature ° C.” and the discomfort factor “air conditioning (hot)” of “ID; a-1”.

The discomfort zone estimating unit 110 then refers to the most recent environmental information that is stored in the learning database 112 and matches the identification information about the estimation condition acquired in step ST150, and acquires the environmental information of the time at which the action information is detected (step ST151). The discomfort zone estimating unit 110 also acquires the time stamp corresponding to the environmental information acquired in step ST151, as the discomfort zone (step ST152).

For example, when referring to the learning database 112 shown in FIG. 5, the discomfort zone estimating unit 110 acquires “temperature 28° C.” as the environmental information of the time at which the action pattern is detected, from “temperature 28° C., noise 35 dB”, which is the environmental information 112b in the most recent history information, on the basis of the estimation condition acquired in step ST150. The discomfort zone estimating unit 110 also acquires the time stamp “2016/8/1/11:04:30” of the acquired environmental information as the discomfort zone.

The discomfort zone estimating unit 110 refers to environmental information in the history information stored in the learning database 112 (step ST153), and determines whether the environmental information in the history information matches the environmental information of the time at which the action pattern acquired in step ST151 is detected (step ST154). If the environmental information in the history information matches the environmental information of the time at which the action pattern is detected (step ST154; YES), the discomfort zone estimating unit 110 adds the time indicated by the time stamp of the matching history information to the discomfort zone (step ST155). The discomfort zone estimating unit 110 determines whether all the environmental information in the history information stored in the learning database 112 has been referred to (step ST156).

If not all the environmental information in the history information has not been referred to yet (step ST156; NO), the operation returns to the process in step ST153, and the above described processes are repeated. If all the environmental information in the history information has been referred to (step ST156; YES), on the other hand, the discomfort zone estimating unit 110 outputs the discomfort zone added in step ST155 as the estimated discomfort zone to the learning unit 109 (step ST157). The discomfort zone estimating unit 110 also outputs the discomfort factor acquired in step ST150 to the learning unit 109.

For example, in a case where the learning database 112 shown in FIG. 5 is referred to, the time from “2016/8/1/11:01:00” to “2016/8/1/11:04:30” indicated by the time stamp of the history information matching “temperature 28° C.” acquired as the discomfort zone estimation condition is output as the discomfort zone to the learning unit 109. After that, the operation proceeds to the process in step ST141 in the flowchart in FIG. 7.

In the above described step ST154, the discomfort zone estimating unit 110 determines whether environmental information in the history information matches the environmental information of the time at which the action pattern is detected. However, a check may be made to determine whether the environmental information falls within a threshold range that is set on the basis of the environmental information of the time at which the action pattern is detected. For example, in a case where the environmental information of the time at which the action pattern is detected is “28° C.”, the discomfort zone estimating unit 110 sets “lower limit: 27.5° C., upper limit: none” as the threshold range. The discomfort zone estimating unit 110 adds the time indicated by the time stamp of the history information within the range to the discomfort zone.

For example, as shown in FIG. 18D, the continuous zone from “2016/8/1/11:01:00” to “2016/8/1/11:04:30”, which indicates a temperature equal to or higher than the lower limit of the threshold range, is estimated as the discomfort zone.

In the flowchart in FIG. 14, the learning unit 109 refers to the learning database 112, and extracts the reaction patterns stored in the discomfort zone estimated in step ST140 as discomfort reaction pattern candidates A (step ST141).

For example, when referring to the learning database 112 shown in FIG. 5, the learning unit 109 extracts the reaction pattern IDs “b-1”, “b-2”, “b-3”, and “b-4” in the zone from “2016/8/1/11:01:00” to “2016/8/1/11:04:30”, which is the estimated discomfort zone, as the discomfort reaction pattern candidates A.

The learning unit 109 then refers to the learning database 112, and learns the discomfort reaction pattern candidate in a zone having environmental information similar to the discomfort zone estimated in step ST140 (step ST142).

FIG. 16 is a flowchart showing an operation of the learning unit 109 of the state estimation device 100 according to the first embodiment, and is a flowchart showing the process in step ST142 in detail.

The learning unit 109 refers to the learning database 112, and searches for a zone in which environmental information is similar to the discomfort zone estimated in step ST140 (step ST160).

As shown in FIG. 18E, for example, through the search process in step ST160, the learning unit 109 acquires a zone that matches the temperature condition in the past, such as a zone (from time t1 to time t2) in which the temperature information stayed at 28° C.

Alternatively, through the search process in step ST160, the learning unit 109 may acquire a zone in which the temperature condition is within a preset range (a range of 27.5° C. and higher) in the past.

The learning unit 109 refers to the learning database 112, and determines whether reaction pattern IDs are stored in the zone searched for in step ST160 (step ST161). If any reaction pattern ID is not stored (step ST161; NO), the operation proceeds to the process in step ST163. If reaction pattern IDs are stored (step ST161; YES), on the other hand, the learning unit 109 extracts the reaction pattern IDs as discomfort reaction pattern candidates B (step ST162).

For example, as shown in FIG. 18E, the reaction pattern IDs “b-1”, “b-2”, and “b-3” stored in the searched zone from time t1 to time t2 are extracted as the discomfort reaction pattern candidates B.

The learning unit 109 then determines whether all the history information in the learning database 112 has been referred to (step ST163). If not all the history information has not been referred to (step ST163; NO), the operation returns to the process in step ST160. If all the history information has been referred to (step ST163; YES), on the other hand, the learning unit 109 excludes a reaction pattern with a low appearance frequency from the discomfort reaction pattern candidates A extracted in step ST141 and the discomfort reaction pattern candidates B extracted in step ST162 (step ST164). The learning unit 109 then sets the eventual discomfort reaction pattern candidates that are the reaction patterns from which a reaction pattern ID with a low appearance frequency has been excluded in step ST164. After that, the operation proceeds to the process in step ST143 in the flowchart in FIG. 14.

In the example shown in FIG. 18F, the learning unit 109 compares the reaction pattern IDs “b-1”, “b-2”, “b-3”, and “b-4” extracted as the discomfort reaction pattern candidates A with the reaction pattern IDs “b-1”, “b-2”, and “b-3” extracted as the discomfort reaction pattern candidates B, and excludes the reaction pattern ID “b-4” included only among the discomfort reaction pattern candidates A as the pattern ID with a low appearance frequency.

In the flowchart in FIG. 14, the learning unit 109 refers to the learning database 112, and learns a reaction pattern at a time when the user is not in an uncomfortable state during a zone having an environmental condition not similar to the discomfort zone estimated in step ST140 (step ST143).

FIG. 17 is a flowchart showing an operation of the learning unit 109 of the state estimation device 100 according to the first embodiment, and is a flowchart showing the process in step ST143 in detail.

The learning unit 109 refers to the learning database 112, and searches for a past zone having environmental information not similar to the discomfort zone estimated in step ST140 (step ST170). Specifically, the learning unit 109 searches for a zone in which environmental information does not match or a zone in which environmental information is outside the preset range.

In the example shown in FIG. 18G, the learning unit 109 searches for the zone (from time t3 to time t4) in which the temperature information stayed “lower than 28° C.” in the past as a zone with environmental information not similar to the discomfort zone.

The learning unit 109 refers to the learning database 112, and determines whether a reaction pattern ID is stored in the zone searched for in step ST170 (step ST171). If any reaction pattern ID is not stored (step ST171; NO), the operation proceeds to the process in step ST173. If a reaction pattern ID is stored (step ST171; YES), on the other hand, the learning unit 109 extracts the stored reaction pattern ID as a non-discomfort reaction pattern candidate (step ST172).

In the example shown in FIG. 18G, the pattern ID “b-2” stored in the zone (from time t3 to time t4) in which the temperature information stayed “lower than 28° C.” in the past is extracted as a non-discomfort reaction pattern candidate.

The learning unit 109 then determines whether all the history information in the learning database 112 has been referred to (step ST173). If not all the history information has not been referred to (step ST173; NO), the operation returns to the process in step ST170. If all the history information has been referred to (step ST173; YES), on the other hand, the learning unit 109 excludes a reaction pattern with a low appearance frequency among the non-discomfort reaction pattern candidates extracted in step ST172 (step ST174). The learning unit 109 then sets the eventual non-discomfort reaction patterns that are the reaction patterns from which a reaction pattern with a low appearance frequency has been excluded in step ST174. After that, the operation proceeds to the process in step ST144 in FIG. 14.

In the example shown in FIG. 18G, if the ratio between the number of extracted pattern IDs “b-2” extracted as the non-discomfort reaction pattern candidate and the number of zones extracted as zones having environmental information not similar to the discomfort zone is lower than a threshold, the reaction pattern ID “b-2” is excluded from the non-discomfort reaction pattern candidates. Note that, in the example shown in FIG. 18G, the reaction pattern ID “b-2” is not excluded.

In the flowchart in FIG. 14, the learning unit 109 excludes the non-discomfort reaction pattern learned in step ST143 from the discomfort reaction pattern candidates learned in step ST142, and acquires a discomfort reaction pattern (step ST144).

In the example shown in FIG. 18H, the reaction pattern ID “b-2”, which is a non-discomfort reaction pattern candidate, is excluded from the reaction pattern IDs “b-1”, “b-2”, and “b-3”, which are the discomfort reaction pattern candidates, and acquires the reaction pattern IDs “b-1” and “b-3” after the exclusion as a discomfort reaction pattern.

The learning unit 109 stores the discomfort reaction pattern acquired in step ST144, together with the discomfort factor input from the discomfort zone estimating unit 110, into the discomfort reaction pattern database 111 (step ST145).

In the example shown in FIG. 4, the learning unit 109 stores the reaction pattern IDs “b-1” and “b-3” extracted as discomfort reaction patterns, together with a discomfort factor “air conditioning (hot)”. After that, the flowchart returns to the process in step ST101 in FIG. 7.

Next, the above mentioned process in step ST135 in the flowchart in FIG. 13 is described in detail.

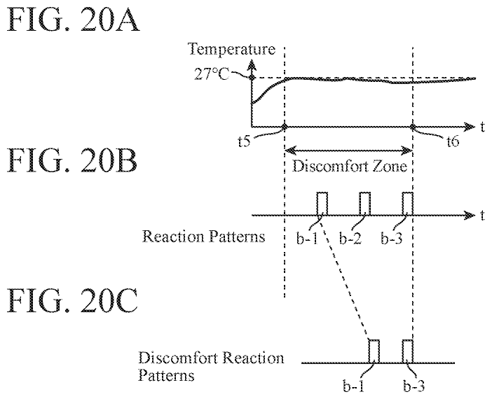

The following description will be made with reference to the examples of storage in the databases shown in FIGS. 2 through 5, a flowchart shown in FIG. 19, and an example of uncomfortable state estimation shown in FIG. 20.

FIG. 19 is a flowchart showing an operation of the discomfort determining unit 108 of the state estimation device 100 according to the first embodiment.

FIG. 20 is a diagram showing an example of uncomfortable state estimation by the state estimation device 100 according to the first embodiment.

The discomfort determining unit 108 refers to the discomfort reaction pattern database 111, and determines whether any discomfort reaction pattern is stored (step ST180). If any discomfort reaction pattern is not stored (step ST180; NO), the operation proceeds to the process in step ST190.

If a discomfort reaction pattern is stored (step ST180; YES), on the other hand, the discomfort determining unit 108 compares the stored discomfort reaction pattern with the identification information about the reaction pattern input from the reaction detecting unit 106 in step ST127 of FIG. 12 (step ST181). A check is made to determine whether the discomfort reaction pattern includes the identification information about the reaction pattern detected by the reaction detecting unit 106 (step ST182). If the identification information about the reaction pattern is not included (step ST182; NO), the discomfort determining unit 108 proceeds to the process in step ST189. If the identification information about the reaction pattern is included (step ST182; YES), on the other hand, the discomfort determining unit 108 refers to the discomfort reaction pattern database 111, and acquires the discomfort factor associated with the identification information about the reaction pattern (step ST183). The discomfort determining unit 108 acquires, from the environmental information acquiring unit 101, the environmental information of the time at which the discomfort factor is acquired in step ST183 (step ST184). The discomfort determining unit 108 estimates a discomfort zone from the acquired environmental information (step ST185).

In the example shown in FIG. 20A, when the reaction pattern ID “b-3” is input from the reaction detecting unit 106 in the case of the storage example shown in FIG. 4, the discomfort determining unit 108 acquires environmental information (temperature information: 27° C.) of the time at which the ID “b-3” is acquired. The discomfort determining unit 108 refers to the learning database 112, and estimates a discomfort zone that is the past zone (from time t5 to time t6) until the temperature information becomes lower than 27° C.

The discomfort determining unit 108 refers to the learning database 112, and extracts the identification information about the reaction patterns detected in the discomfort zone estimated in step ST185 (step ST186). The discomfort determining unit 108 determines whether the identification information about the reaction patterns extracted in step ST186 matches the discomfort reaction patterns stored in the discomfort reaction pattern database 111 (step ST187). If a matching discomfort reaction pattern is stored (step ST187; YES), the discomfort determining unit 108 estimates that the user is in an uncomfortable state (step ST188).

In the example shown in FIG. 20B, the discomfort determining unit 108 extracts the reaction pattern IDs “b-1”, “b-2”, and “b-3” detected in the estimated discomfort zone.

The discomfort determining unit 108 determines whether the reaction pattern IDs “b-1”, “b-2”, and “b-3” in FIG. 20B match the discomfort reaction patterns stored in the discomfort reaction pattern database 111 in FIG. 20C.

In the case of the example of storage in the discomfort reaction pattern database 111 shown in FIG. 4, all the discomfort reaction pattern IDs “b-1” and “b-3” in a case where the discomfort factor 111a is “air conditioning (hot)” are included among the extracted reaction pattern IDs. In this case, the discomfort determining unit 108 determines that a matching discomfort reaction pattern is stored in the discomfort reaction pattern database 111, and estimates that the user is in an uncomfortable state.

If any matching discomfort reaction pattern is not stored (step ST187; NO), on the other hand, the discomfort determining unit 108 determines whether checking against all the discomfort reaction patterns has been completed (step ST189). If checking against all the discomfort reaction patterns has not been completed yet (step ST189; NO), the operation returns to the process in step ST181. If checking against all the discomfort reaction patterns has been completed (step ST189; YES), on the other hand, the discomfort determining unit 108 estimates that the user is not in an uncomfortable state (step ST190). If the process in step ST188 or step ST190 has been performed, the flowchart proceeds to the process in step ST136 in FIG. 13.

As described above, the state estimation device according to the first embodiment includes: the action detecting unit 104 that checks at least one piece of behavioral information including motion information about a user, sound information about the user, and operation information about the user against action patterns stored in advance, and detects a matching action pattern; the reaction detecting unit 106 that checks the behavioral information and biological information about the user against reaction patterns stored in advance, and detects a matching reaction pattern; the discomfort determining unit 108 that determines that the user is in an uncomfortable state in a case where a matching action pattern has been detected, or where a matching reaction pattern has been detected and the reaction pattern matches a discomfort reaction pattern indicating an uncomfortable state of the user, the discomfort reaction pattern being stored in advance; the discomfort zone estimating unit 110 that acquires an estimation condition for estimating a discomfort zone on the basis of a detected action pattern, and estimates a discomfort zone that is the zone matching the acquired estimation condition in history information stored in advance; and the learning unit 109 that refers to the history information, and acquires and stores a discomfort reaction pattern on the basis of the estimated discomfort zone and the occurrence frequencies of reaction patterns in the zones other than the discomfort zone. With this configuration, it is possible to determine whether a user is in an uncomfortable state, and estimate the state of the user, without the user inputting information about his/her uncomfortable state or a discomfort factor corresponding to a reaction not associated directly with any discomfort factor. Thus, user-friendliness can be increased.

Further, even in a state where a large amount of history information is not accumulated, it is possible to acquire and store a discomfort reaction pattern through learning. Thus, it is possible to estimate a user state without taking a long time from the start of use of the state estimation device and improve user-friendliness.

Also, according to the first embodiment, the learning unit 109 extracts discomfort reaction pattern candidates on the basis of the occurrence frequencies of the reaction patterns in the history information in a discomfort zone, extracts non-discomfort reaction patterns on the basis of the occurrence frequencies of the reaction patterns in the history information in the zones other than the discomfort zone, and acquires discomfort reaction patterns that are reaction patterns obtained by excluding the non-discomfort reaction patterns from the discomfort reaction patterns. With this configuration, an uncomfortable state can be determined from only the reaction patterns the user is highly likely to show depending on a discomfort factor, and the reaction patterns the user is highly likely to show regardless of discomfort factors can be excluded from the reaction patterns to be used in determining an uncomfortable state. Thus, the accuracy of uncomfortable state estimation can be increased.

Further, according to the first embodiment, the discomfort determining unit 108 determines that the user is in an uncomfortable state, in a case where a matching reaction pattern has been detected by the reaction detecting unit 106, and the detected reaction pattern matches a discomfort reaction pattern that is stored in advance and indicates an uncomfortable state of the user. With this configuration, it is possible to estimate an uncomfortable state of the user before the user takes an action associated directly with a discomfort factor, and cause an external device to perform control to remove the discomfort factor. Because of this, user-friendliness can be increased.

In the first embodiment described above, the environmental information acquiring unit 101 acquires temperature information detected by a temperature sensor, and noise information indicating the magnitude of noise collected by a microphone. However, humidity information detected by a humidity sensor and information about brightness detected by an illuminance sensor may be acquired. Alternatively, the environmental information acquiring unit 101 may acquire humidity information and brightness information, in addition to the temperature information and the noise information. Using the humidity information and the brightness information acquired by the environmental information acquiring unit 101, the state estimation device 100 can estimate that the user is in an uncomfortable state due to dryness, a high humidity, a situation that is too bright, or a situation that is too dark.

In the first embodiment described above, the biological information acquiring unit 103 acquires information indicating fluctuations in the user's heart rate measured by a heart rate meter or the like as biological information. However, information indicating fluctuations in the user's brain waves measured by an electroencephalograph attached to the user may be acquired. Alternatively, the biological information acquiring unit 103 may acquire both information indicating fluctuations in the heart rate and information indicating fluctuations in the brain waves as the biological information. Using the information that indicates fluctuations in the brain waves and has been acquired by the biological information acquiring unit 103, the state estimation device 100 can increase the accuracy in estimating the user's uncomfortable state in a case where a change appears in the fluctuations in the brain waves as a reaction pattern at a time when the user feels discomfort.

Further, in a case where action pattern identification information is included in the discomfort zone estimated by the discomfort zone estimating unit 110 in the state estimation device according to the first embodiment described above, if the discomfort factor corresponding to the action pattern identification information does not match the discomfort factor used as the estimation condition for estimating the discomfort zone, the reaction patterns in the zone may not be extracted as discomfort reaction pattern candidates. In this manner, the reaction patterns corresponding to different discomfort factors can be prevented from being erroneously stored as discomfort reaction patterns into the discomfort reaction pattern database 111. Thus, the accuracy of uncomfortable state estimation can be increased.

Further, in the state estimation device according to the first embodiment described above, the discomfort zone estimated by the discomfort zone estimating unit 110 is estimated on the basis of an estimation condition 105d in the action information database 105. Alternatively, the state estimation device may store information about all the device operations of the user into the learning database 112, and excludes the zone in a certain period after a device operation is performed from the discomfort zone candidates. By doing so, it is possible to exclude the reactions that have occurred during the certain period after a user performs a device operation, from the user reactions to device operations. Thus, the accuracy in estimating an uncomfortable state of a user can be increased.

Further, in the state estimation device according to the first embodiment described above, in a zone with environmental information similar to the discomfort zone estimated by the discomfort zone estimating unit 110 on the basis of a discomfort factor, reaction patterns obtained by excluding the reaction patterns with low appearance frequencies are set as the discomfort reaction pattern candidates. Accordingly, only the non-discomfort reaction patterns highly likely to be shown by a user depending on the discomfort factor can be used in estimating an uncomfortable state. Thus, the accuracy in estimating an uncomfortable state of a user can be increased.

Further, in the state estimation device according to the first embodiment described above, in a zone with environmental information not similar to the discomfort zone estimated by the discomfort zone estimating unit 110 on the basis of a discomfort factor, reaction patterns obtained by excluding the reaction patterns with high appearance frequencies are set as the discomfort reaction pattern candidates. Accordingly, the non-discomfort reaction patterns highly likely to be shown by a user regardless of the discomfort factor can be excluded from those to be used in estimating an uncomfortable state. Thus, the accuracy in estimating an uncomfortable state of a user can be increased.

Note that, in the state estimation device according to the first embodiment described above, when operation information is included in the action pattern detected by the action detecting unit 104, the discomfort zone estimating unit 110 may exclude the zone in a certain period after the acquisition of the operation information, from the discomfort zone.

By doing so, it is possible to exclude the reactions occurring during the certain period after the device changes the upper limit temperature of the air conditioner as the user's reactions to control of the device, for example. Thus, the accuracy in estimating an uncomfortable state of a user can be increased.

### Second Embodiment

A second embodiment concerns a configuration for changing the methods of estimating a user's uncomfortable state, depending on the amount of the history information accumulated in the learning database 112.

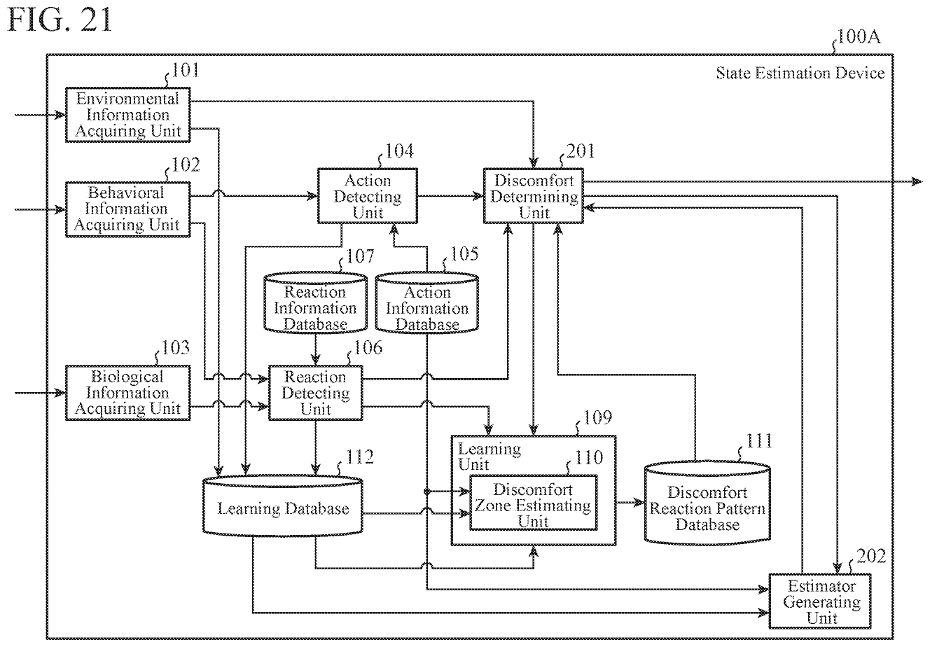

FIG. 21 is a block diagram showing the configuration of a state estimation device 100A according to the second embodiment.

The state estimation device 100A according to the second embodiment includes a discomfort determining unit 201 in place of the discomfort determining unit 108 of the state estimation device 100 according to the first embodiment shown in FIG. 1, and further includes an estimator generating unit 202.

In the description below, the components that are the same as or equivalent to the components of the state estimation device 100 according to the first embodiment are denoted by the same reference numerals as the reference numerals used in the first embodiment, and are not explained or are only briefly explained.

In a case where an estimator is generated by the estimator generating unit 202 described later, the discomfort determining unit 201 estimates an uncomfortable state of a user, using the generated estimator. In a case where any estimator is not generated by the estimator generating unit 202, the discomfort determining unit 201 estimates an uncomfortable state of the user, using the discomfort reaction pattern database 111.

In a case where the number of action patterns in the history information stored in the learning database 112 becomes equal to or larger than a prescribed value, the estimator generating unit 202 performs machine learning using the history information stored in the learning database 112. Here, the prescribed value is a value that is set on the basis of the number of action patterns necessary for the estimator generating unit 202 to generate an estimator. The estimator generating unit 202 performs machine learning. In the machine learning, input signals are the reaction patterns and environmental information extracted for the respective discomfort zones estimated from the identification information about action patterns, and output signals are information indicating a comfortable state or an uncomfortable state of a user with respect to each of the discomfort factors corresponding to the identification information about the action patterns. The estimator generating unit 202 generates an estimator for estimating a user's uncomfortable state from a reaction pattern and environmental information. The machine learning to be performed by the estimator generating unit 202 is performed by applying the deep learning method described in Non-Patent Literature 1 shown below, for example.

### Non-Patent Literature 1

Takayuki Okaya, “Deep Learning”, Journal of the Institute of Image Information and Television Engineers, Vol. 68, No. 6, 2014

Next, an example hardware configuration of the state estimation device 100A is described. Note that explanation of the same components as those of the first embodiment is not made herein.

The discomfort determining unit 201 and the estimator generating unit 202 in the state estimation device 100A are the processing circuit 100a shown in FIG. 6A, or are the processor 100b that executes programs stored in the memory 100c shown in FIG. 6B.

Next, operation of the estimator generating unit 202 is described.

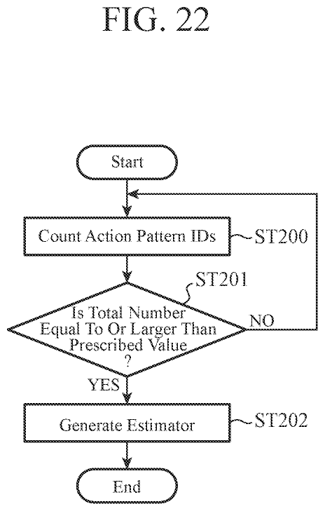

FIG. 22 is a flowchart showing an operation of the estimator generating unit 202 of the state estimation device 100A according to the second embodiment.

The estimator generating unit 202 refers to the learning database 112 and the action information database 105, and counts the action pattern IDs stored in the learning database 112 for each discomfort factor (step ST200). The estimator generating unit 202 determines whether the total number of the action pattern IDs counted in step ST200 is equal to or larger than a prescribed value (step ST201). If the total number of the action pattern IDs is smaller than the prescribed value (step ST201; NO), the operation returns to the process in step ST200, and the above described process is repeated.

If the total number of the action pattern IDs is equal to or larger than the prescribed value (step ST201; YES), on the other hand, the estimator generating unit 202 performs machine learning, and generates an estimator for estimating a user's uncomfortable state from a reaction pattern and environmental information (step ST202). After the estimator generating unit 202 generates an estimator in step ST202, the process comes to an end.

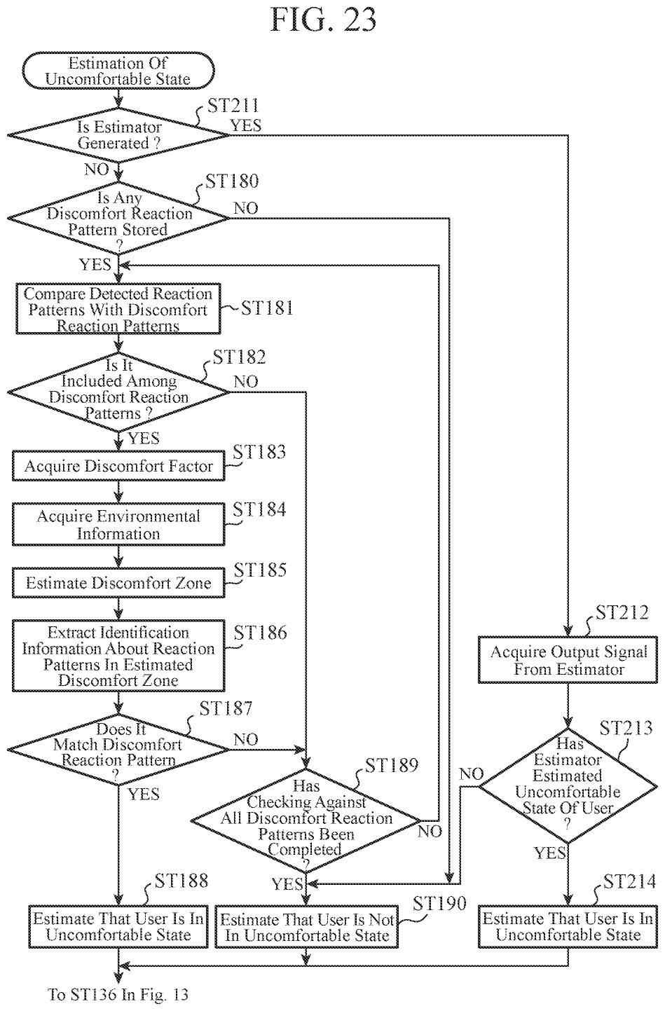

FIG. 23 is a flowchart showing an operation of the discomfort determining unit 201 of the state estimation device 100A according to the second embodiment.

In FIG. 23, the same steps as those in the flowchart of the first embodiment shown in FIG. 19 are denoted by the same reference numerals as those used in FIG. 19, and explanation of them is not made herein.

The discomfort determining unit 201 refers to the state of the estimator generating unit 202, and determines whether an estimator is generated (step ST211). If an estimator is generated (step ST211; YES), the discomfort determining unit 201 inputs a reaction pattern and environmental information as input signals to the estimator, and acquires a result of estimation of a user's uncomfortable state as an output signal (step ST212). The discomfort determining unit 201 refers to the output signal acquired in step ST212, and determines whether or the estimator has estimated an uncomfortable state of the user (step ST213). When the estimator has estimated an uncomfortable state of the user (step ST213; YES), the discomfort determining unit 201 estimates that the user is in an uncomfortable state (step ST214).

If any estimator has not been generated (step ST211; NO), on the other hand, the discomfort determining unit 201 refers to the discomfort reaction pattern database 111, and determines whether any discomfort reaction pattern is stored (step ST180). After that, the processes from step ST181 to step ST190 are performed. If the process in step ST188, step ST190, or step ST214 has been performed, the flowchart proceeds to the process in step ST136 in FIG. 13.

As described above, according to the second embodiment, the state estimation device includes the estimator generating unit 202 that generates an estimator for estimating whether a user is in an uncomfortable state, on the basis of a reaction pattern detected by the reaction detecting unit 106 and environmental information in a case where the number of the action patterns accumulated as history information is equal to or larger than a prescribed value. In a case where an estimator is generated, the discomfort determining unit 201 determines whether the user is in an uncomfortable state, by referring to the result of the estimation by the estimator. With this configuration, in a case where the number of the action patterns in the history information is smaller than the prescribed value, an uncomfortable state of the user and a discomfort factor can be estimated on the basis of the discomfort reaction patterns stored in the discomfort reaction pattern database. In a case where the number of the action patterns is equal to or larger than the prescribed value, an uncomfortable state of the user and a discomfort factor can be estimated with an estimator generated through machine learning. By virtue of this, the accuracy in estimating an uncomfortable state of a user can be increased.

Note that, in the second embodiment described above, the estimator generating unit 202 performs machine learning, using input signals that are the reaction patterns stored in the learning database 112. In addition to this, information not registered in the action information database 105 and the reaction information database 107 may be stored into the learning database 112, and the stored information may be used as input signals in the machine learning. This makes it possible to learn users' habits that are not registered in the action information database 105 and the reaction information database 107, and the accuracy in estimating an uncomfortable state of a user can be increased.

### Third Embodiment

A third embodiment concerns a configuration for estimating a discomfort factor as well as an uncomfortable state, from a detected reaction pattern.

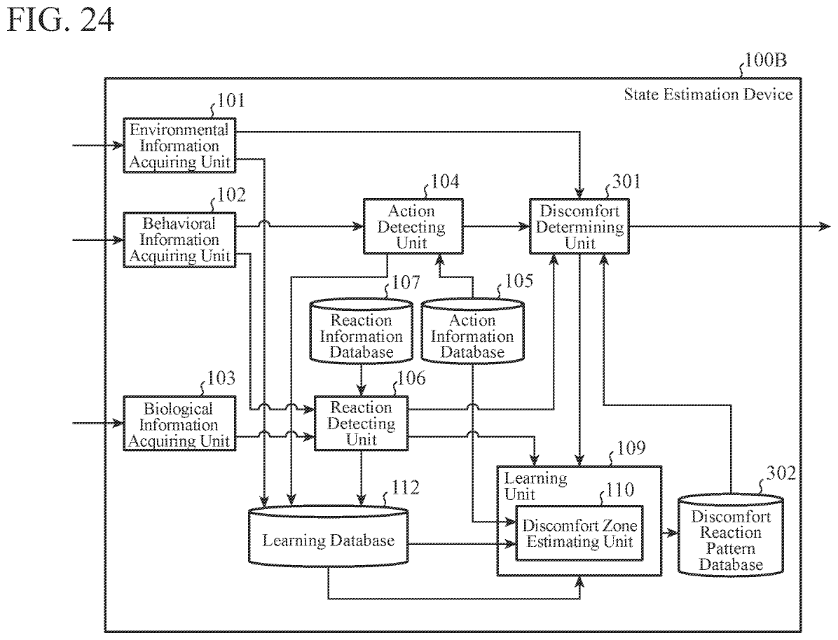

FIG. 24 is a block diagram showing the configuration of a state estimation device 100B according to the third embodiment.

The state estimation device 100B according to the third embodiment includes a discomfort determining unit 301 and a discomfort reaction pattern database 302, in place of the discomfort determining unit 108 and the discomfort reaction pattern database 111 of the state estimation device 100 of the first embodiment shown in FIG. 1.

In the description below, the components that are the same as or equivalent to the components of the state estimation device 100 according to the first embodiment are denoted by the same reference numerals as the reference numerals used in the first embodiment, and are not explained or are only briefly explained.

When the identification information about a detected reaction pattern is input from the reaction detecting unit 106, the discomfort determining unit 301 checks the input identification information against the discomfort reaction patterns that are stored in the discomfort reaction pattern database 302 and indicate uncomfortable states of users. In a case where a reaction pattern matching the input identification information is stored in the discomfort reaction pattern database 302, the discomfort determining unit 301 estimates that the user is in an uncomfortable state. The discomfort determining unit 301 further refers to the discomfort reaction pattern database 302, and, in a case where the discomfort factor can be identified from the input identification information, identifies the discomfort factor. The discomfort determining unit 301 outputs a signal indicating that an uncomfortable state of the user has been detected, and, in a case where the discomfort factor has been successfully identified, outputs a signal indicating information about the discomfort factor to the outside.

The discomfort reaction pattern database 302 is a database that stores discomfort reaction patterns that are the results of learning by the learning unit 109.

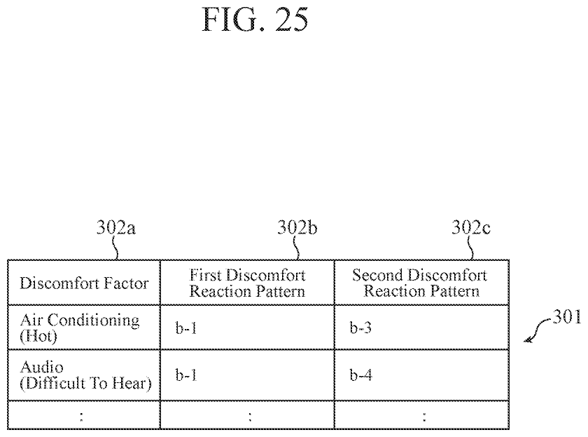

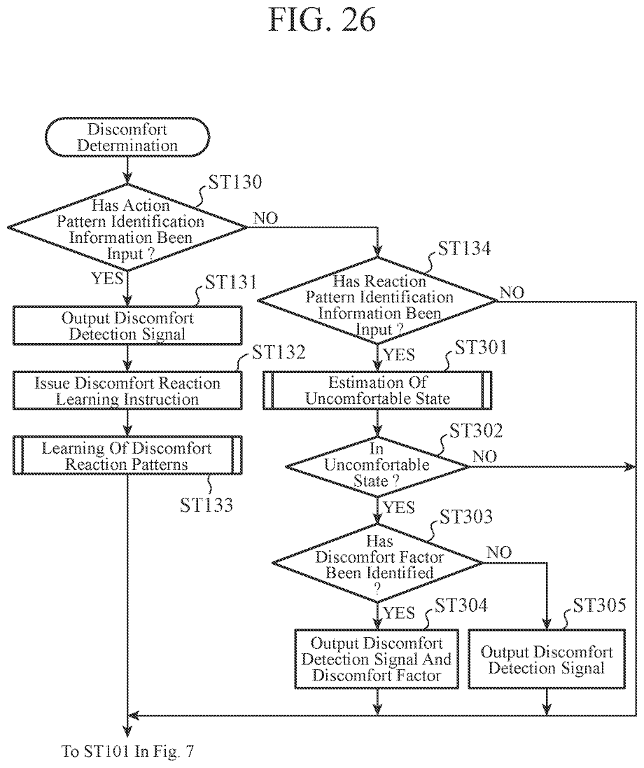

FIG. 25 is a table showing an example of storage in the discomfort reaction pattern database 302 of the state estimation device 100B according to the third embodiment.

The discomfort reaction pattern database 302 shown in FIG. 25 contains the following items: discomfort factors 302a, first discomfort reaction patterns 302b, and second discomfort reaction patterns 302c. The same items as the items of the discomfort factors 105b in the action information database 105 (see FIG. 2) are written as the discomfort factors 302a. The ID of a discomfort reaction pattern corresponding to more than one discomfort factor 302a is written as the first discomfort reaction patterns 302b. The IDs of discomfort reaction patterns each corresponding to a particular discomfort factor are written as the second discomfort reaction patterns 302c. The IDs of the discomfort reaction patterns written as the first and second discomfort reaction patterns 302b and 302c correspond to the IDs 107a shown in FIG. 3.

In a case where input identification information matches the identification information about a second discomfort reaction pattern 302c, the discomfort determining unit 301 acquires the discomfort factor 302a associated with the matching identification information. Thus, the discomfort factor is identified.

An example hardware configuration of the state estimation device 100B is now described. Note that explanation of the same components as those of the first embodiment is not made herein.

The discomfort determining unit 301 and the discomfort reaction pattern database 302 in the state estimation device 100B are the processing circuit 100a shown in FIG. 6A, or are the processor 100b that executes programs stored in the memory 100c shown in FIG. 6B.

Next, operation of the discomfort determining unit 301 is described.

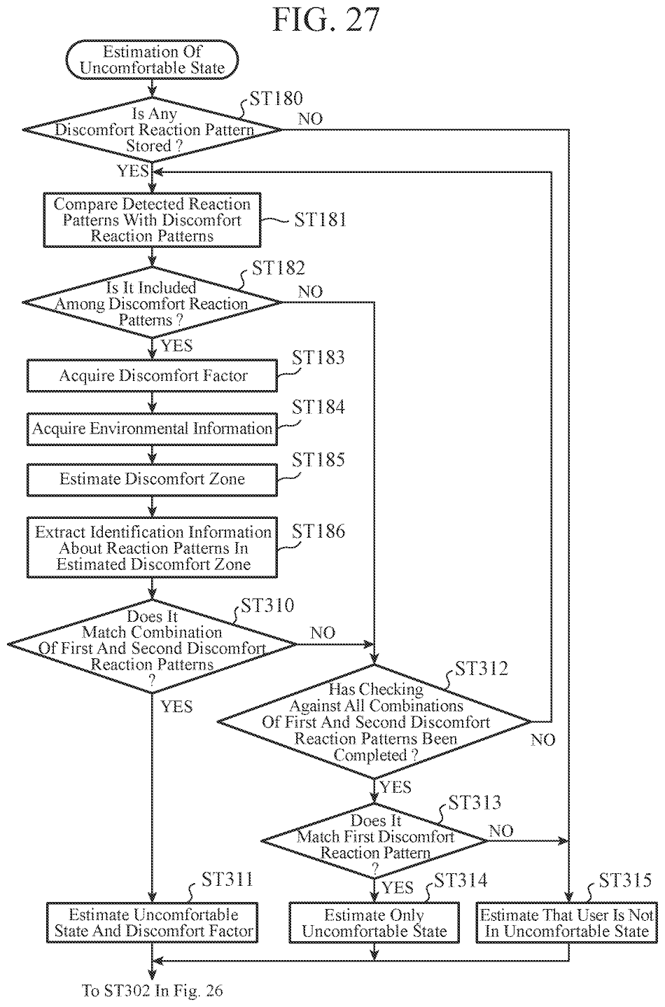

FIG. 26 is a flowchart showing an operation of the discomfort determining unit 301 of the state estimation device 100B according to the first embodiment.

In FIG. 26, the same steps as those in the flowchart of the first embodiment shown in FIG. 13 are denoted by the same reference numerals as those used in FIG. 13, and explanation of them is not made herein.

If the discomfort determining unit 301 determines in step ST134 that the identification information about a reaction pattern has been input (step ST134; YES), the discomfort determining unit 301 checks the input identification information about the reaction pattern against the first discomfort reaction patterns 302b and the second discomfort reaction patterns 302c stored in the discomfort reaction pattern database 302, and estimates an uncomfortable state of the user (step ST301). The discomfort determining unit 301 refers to the result of the estimation in step ST301, and determines whether the user is in an uncomfortable state (step ST302).

If the user is determined to be in an uncomfortable state (step ST302; YES), the discomfort determining unit 301 refers to the result of the checking, and determines whether the discomfort factor has been identified (step ST303). If the discomfort factor has been identified (step ST303; YES), the discomfort determining unit 301 outputs, to the outside, a signal indicating that an uncomfortable state of the user has been detected, together with the discomfort factor (step ST304). If any discomfort factor has not been identified (step ST303; NO), on the other hand, the discomfort determining unit 301 outputs, to the outside, a signal indicating that the discomfort factor is unknown, but an uncomfortable state of the user has been detected (step ST305).

If the process in step ST133 has been performed, if the process in step ST304 has been performed, if the process in step ST305 has been performed, if any identification information about any reaction pattern has not been input (step ST134; NO), or if the user is determined not to be in an uncomfortable state (step ST302; NO), the flowchart returns to the process in step ST101 in FIG. 7.

Next, the above mentioned process in step ST301 in the flowchart in FIG. 26 is described in detail.

FIG. 27 is a flowchart showing an operation of the discomfort determining unit 301 of the state estimation device 100B according to the third embodiment.

In FIG. 27, the same steps as those in the flowchart of the first embodiment shown in FIG. 19 are denoted by the same reference numerals as those used in FIG. 19, and explanation of them is not made herein.

After extracting the identification information about reaction patterns in step ST186, the discomfort determining unit 301 determines whether the extracted identification information about the reaction patterns matches a combination of the first and second discomfort reaction patterns (step ST310). If it is determined to match a combination of the first and second discomfort reaction patterns (step ST310; YES), the discomfort determining unit 301 estimates that it is in an uncomfortable state, and estimates the discomfort factor (step ST311). If it is determined not to match any combination of the first and second discomfort reaction patterns (step ST310: NO), on the other hand, the discomfort determining unit 301 determines whether checking against all the combinations of the first and second discomfort reaction patterns has been completed (step ST312).

If checking against all the combinations of the first and second discomfort reaction patterns has not been completed yet (step ST312; NO), the discomfort determining unit 301 returns to the process in step ST181. If checking against all the combinations of the first and second discomfort reaction patterns has been completed (step ST312; YES), on the other hand, the discomfort determining unit 301 determines whether the identification information about the reaction pattern matches a first discomfort reaction pattern (step ST313). If the identification information matches a first discomfort reaction pattern (step ST313; YES), the discomfort determining unit 301 estimates that it is in an uncomfortable state (step ST314). In the process in step ST314, only an uncomfortable state is estimated, and the discomfort factor is not estimated.

If the identification information does not match any first discomfort reaction pattern (step ST313; NO), on the other hand, the discomfort determining unit 301 estimates that it is not in an uncomfortable state (step ST315). If the discomfort determining unit 301 determines in step ST180 that any discomfort reaction pattern is not stored (step ST180; NO), the operation also proceeds to the process in the step ST315.

If the process in step ST311, step ST314, or step ST315 has been performed, the flowchart proceeds to the process in step ST302 in FIG. 26.

As described above, according to the third embodiment, in a case where reaction patterns detected by the reaction detecting unit 106 matches stored discomfort reaction patterns, and the reaction pattern corresponding to a particular discomfort factor is included among the matching reaction patterns, the discomfort determining unit 301 identifies the discomfort factor from the reaction pattern corresponding to the particular discomfort factor. Accordingly, in a case where a discomfort factor can be identified, the identified discomfort factor can be promptly removed. Further, in a case where the discomfort factor is unknown, a signal to that effect is output, to inquire of the user about the discomfort factor, for example. In this manner, the discomfort factor can be quickly identified and removed. Thus, the user's comfort can be increased.

Note that, in the third embodiment described above, in a case where matching with the first discomfort reaction pattern corresponding to more than one discomfort factor is detected, the discomfort determining unit 301 promptly estimates that the user is in an uncomfortable state, though the discomfort factor is unknown. However, a timer that operates only in a case where matching with a first discomfort reaction pattern corresponding to more than one discomfort factor is detected. In a case where the matching with the first discomfort reaction pattern lasts for a certain period of time or longer, the discomfort determining unit 301 may estimate that the user is in an uncomfortable state, though the discomfort factor is unknown. This can prevent frequent inquiries to the user about discomfort factors. Thus, the user's comfort can be increased.

Note that, in addition of the above, the embodiments can be freely combined, modifications may be made to any component of each embodiment, or a desired component may be omitted from each embodiment, within the scope of the present invention.

### INDUSTRIAL APPLICABILITY

A state estimation device according to the present invention can estimate a state of a user, without the user inputting information indicating his/her emotional state. Accordingly, the state estimation device is suitable for estimating a user state while reducing the burden on the user in an environmental control system or the like.

### REFERENCE SIGNS LIST

100, 100A, 100B: State estimation device, 101: Environmental information acquiring unit, 102: Behavioral information acquiring unit, 103: Biological information acquiring unit, 104: Action detecting unit, 105: Action information database, 106: Reaction detecting unit, 107: Reaction information database, 108, 201, 301: Discomfort determining unit, 109: Learning unit, 110: Discomfort zone estimating unit, 111, 302: Discomfort reaction pattern database, 112: Learning database, and 202: Estimator generating unit.

1. A state estimation device comprising:
a processor; and
a memory storing instructions which, when executed by the processor, causes the processor to perform processes of:
checking at least one piece of behavioral information including motion information about a user, sound information about the user, and operation information about the user against action patterns stored in advance, and detecting a matching action pattern;
checking the behavioral information and biological information about the user against reaction patterns stored in advance, and detecting a matching reaction pattern;
determining that the user is in an uncomfortable state, when the processor detects a matching action pattern, or when the processor detects a matching reaction pattern and the detected reaction pattern matches a discomfort reaction pattern indicating an uncomfortable state of the user, the discomfort reaction pattern being stored in advance;
acquiring an estimation condition for estimating a discomfort zone on a basis of the detected action pattern, and estimating a discomfort zone, the discomfort zone being a zone matching the acquired estimation condition in history information stored in advance; and
referring to the history information and acquiring and storing the discomfort reaction pattern on a basis of the estimated discomfort zone and an occurrence frequency of a reaction pattern in a zone other than the discomfort zone.

2. The state estimation device according to claim 1, wherein the history information includes at least environmental information about a surrounding of the user, an action pattern of the user, and a reaction pattern of the user.

3. The state estimation device according to claim 2, wherein the processor extracts a discomfort reaction pattern candidate on a basis of an occurrence frequency of a reaction pattern in the history information in the discomfort zone, extracts a non-discomfort reaction pattern on a basis of an occurrence frequency of a reaction pattern in the history information in a zone other than the discomfort zone, and acquires the discomfort reaction pattern from which the non-discomfort reaction pattern is excluded from the discomfort reaction pattern candidate.

4. The state estimation device according to claim 1, wherein, when the detected reaction pattern matches the stored discomfort reaction pattern, and the matching reaction pattern includes a reaction pattern corresponding to a particular discomfort factor, the processor identifies a discomfort factor of the user on a basis of the reaction pattern corresponding to the particular discomfort factor.

5. The state estimation device according to claim 2, further comprising
wherein the processes further include:
generating an estimator for estimating whether the user is in an uncomfortable state, on a basis of the detected reaction pattern and the environmental information, when action patterns equal to or higher than a prescribed value are accumulated as the history information,
wherein, when the estimator is generated, the processor refers to a result of estimation by the estimator and determines whether the user is in an uncomfortable state.

6. The state estimation device according to claim 1, wherein, when the detected action pattern includes the operation information, the processor excludes a zone in a certain period after acquisition of the operation information, from the discomfort zone.
